(19) United States
(12) Patent Application Publication
(10) Pub. No.: US 2010/0130891 A1
(43) Pub. Date: May 27, 2010

(54) Wearable Therapeutic Ultrasound Article

(76) Inventors: Rebecca M. Taggart, Alpharetta, GA (US); Wing-Chak Ng, Suwanee, GA (US); Sridhar Ranganthan, Suwanee, GA (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/275,501

(22) Filed: Nov. 21, 2008

(51) Int. Cl. A61N 7/00

(52) U.S. Cl. 601/2

(57) ABSTRACT

The present invention is directed to a wearable therapeutic article including an ultrasound transducer having a first electrode, a second electrode and a piezoelectric material disposed therebetween, the ultrasound transducer being positioned within a pocket so that, as the user moves, the ultrasound transducer moves to a different position in the pocket.

### BACKGROUND OF THE INVENTION

Ultrasound therapy uses sound waves to enhance healing of soft tissue and bone injuries, and improve pain management by decreasing inflammation. Ultrasound waves are applied to tissue by placing a transducer against the skin. The ultrasound waves can stimulate cell repair by causing the soft tissues to vibrate gently, which may increase nutrient transport into cells, improve the mechanical integrity of cells and increase circulation. During standard ultrasound therapy, an ultrasound transducer probe generates ultrasound waves while it is moved slowly and continuously over the injured region. A gel or other coupling agent may be placed first on the skin to enhance the propagation of the ultrasound waves and to reduce friction. The movement of the ultrasound transducer probe is essential to avoiding the accumulation of acoustic pressure or ‘hot spots’, which are undesirable.

To produce ultrasound waves, an electrical signal is applied to a transducer which may be formed from materials such as quartz, tourmaline or lead zirconate titanate (PZT). The electrical signal causes a mechanical response in the piezoelectric material, known as the reverse piezoelectric effect. A variety of piezoelectric materials may be used to generate sound waves besides a single crystal. Arrays are frequently utilized to produce ultrasound waves. Additionally, robust and flexible piezoelectric fiber composites capable of generating ultrasound waves may also be utilized.

During standard ultrasound therapy applications, a user must remain stationary yet continuously move the ultrasound article over the affected tissue. As such, a need exists for a portable ultrasound therapeutic article which avoids the accumulation of acoustic pressure while permitting a user to move about at will.

### SUMMARY OF THE INVENTION

In accordance with one embodiment of the present invention, a therapeutic article is provided which includes at least one pocket having positioned therein an ultrasound transducer. The ultrasound transducer includes a piezoelectric material which is movable within the pocket. A power supply is in electrical communication with the piezoelectric material so that, as power is supplied to the piezoelectric material an ultrasound wave is produced. As the user moves, the ultrasound transducer remains in electrical communication with the power supply while moving to a different position in the pocket. This results in the application of ultrasound therapy to different points on the tissue.

In accordance with another embodiment of the present invention, an article is provided which includes at least one pocket having interior surfaces, a portion of the interior surfaces being electrically conductive and connected to a power supply. At least one portion of the pocket may be formed from a flexible material. An ultrasound transducer positioned within the pocket may include a first electrode, a second electrode and a piezoelectric material being disposed therebetween, the first and second electrodes in electrical communication with the electrically conductive portions of the interior surfaces of the pocket. The ultrasound transducer is movable within the pocket. A power supply is provided which is in electrical communication with the first and second electrodes. The power supply is preferably portable to permit the user to move about.

Other features and aspects of the present invention are described in more detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a partial perspective view of a therapeutic article in accordance with one embodiment of the present invention; and

FIG. 2 is a partial perspective view of a therapeutic article in accordance with another embodiment of the present invention;

FIG. 3 is a cross-sectional view of the article of FIG. 1 along lines A-A;

FIG. 4 is a cross-sectional view of the article of FIG. 2 along lines B-B; and

FIG. 5 is a perspective view of an embodiment of the present invention on a user.

Repeat use of reference characters in the present specification and drawings is intended to represent same or analogous features or elements of the invention.

### DETAILED DESCRIPTION OF REPRESENTATIVE EMBODIMENTS

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment may be used on another embodiment to yield a still further embodiment. Thus, it is intended that the present invention cover such modifications and variations.

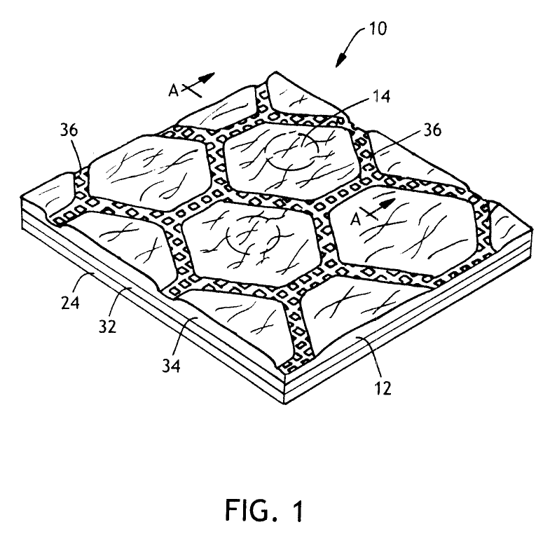

The present invention is generally directed to a therapeutic article suitable for attachment to a user which is capable of providing appropriate ultrasound therapy by advantageously utilizing the movement of the user. As seen in FIGS. 1, 3 and 5, a therapeutic article 10 includes at least one pocket 12 within which is positioned an ultrasound transducer 14. The ultrasound transducer 14 is electrically connected to a portable power source (not shown) in a manner which enables the ultrasound transducer 14 to generate and emit therapeutic ultrasound waves. The ultrasound transducer 14 is free to move within the pocket 12 while it is generating ultrasound waves. Movement of the user causes movement of the ultrasound transducer 14 within the pocket 12. Consistent electrical contact is maintained between the transducer and the power supply while the transducer is in motion.

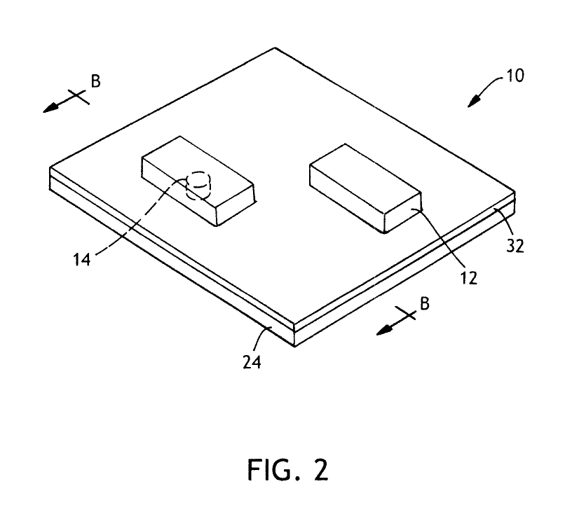

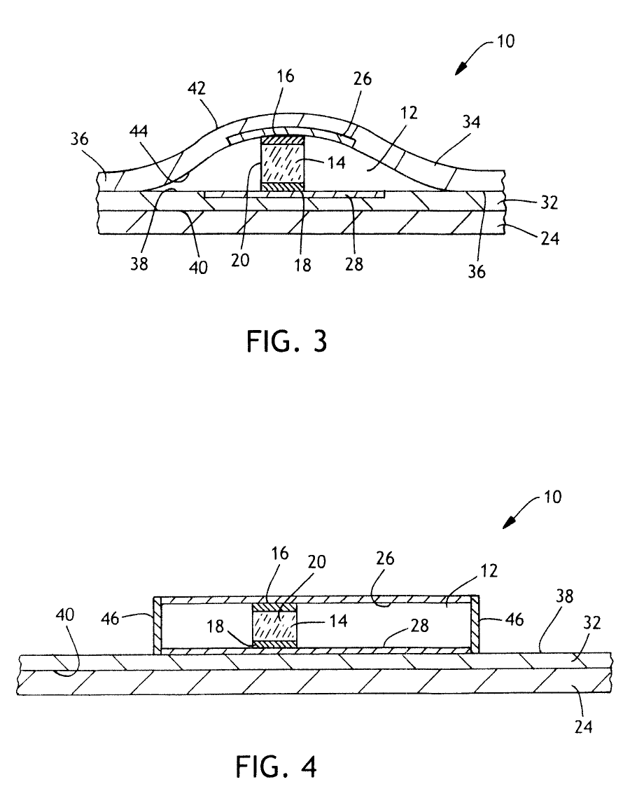

While many different configurations of the article 10 and pocket 12 are possible, the article 10 as seen in FIGS. 2 and 4 include a support layer 32 upon which may be positioned one or more pockets 12. The pockets 12 may be variously configured, and may be formed of rigid materials, flexible materials or combinations of such materials. As seen in FIGS. 1 and 3, the pocket 12 may be formed of an enclosing layer 34 and a support layer 32, at least a portion of the enclosing layer 34 being flexible. The enclosing layer 34 must be sufficiently flexible to permit the ultrasound transducer 14 to move within the pocket 12. In the embodiment shown in FIG. 3, the support layer 32 includes a lower surface 40 and an upper surface 38 which forms a portion of the interior of the pocket 12. A portion of the upper surface 38 may be electrically conductive to permit the pocket 12 to include a conductive lower interior surface 28. The lower surface 40 of the support layer 32 may be attached to an adhesive layer 24, which may also function to enhance the transmission of the ultrasound waves to the user. In the embodiment of FIG. 3, the enclosing layer 34 includes an upper exterior surface 42 and a lower surface 44, the lower surface 44 forming a portion of the interior of the pocket 12. A portion of the lower surface 44 of the enclosing layer 34 may be electrically conductive to permit the pocket 12 to include a conductive upper interior surface 26. The support layer 32 and enclosing layer 34 may be joined together at bond areas 36, as seen in FIGS. 1 and 3.

In the embodiment depicted in FIG. 4, a more rigid pocket 12 is provided which includes interior surfaces 26, 28 which are electrically conductive. The pocket 12 is formed by walls 46. The pocket 12 is joined to the upper surface 38 of the support layer 32. The lower surface 40 of the support layer 32 may be positioned adjacent to a wave transmission layer 24, which may contain and/or be formed of a hydrogel material. The wave transmission layer 24 may also function to attach the article 10 to a user.

As shown in FIGS. 3 and 4, the ultrasound transducer 14 is positioned within the pocket 12. The ultrasound transducer includes a piezoelectric material 20. In some embodiments, the piezoelectric material may be in direct contact with the electrically conductive surfaces 26, 28. In other embodiments, the ultrasound transducer 14 may also include a first electrode 16, a second electrode 18 and a piezoelectric material 20 being disposed therebetween. The first and second electrodes 16, 18 may be positioned on and maintain contact with electrically conductive portions 26 and 28 as the ultrasound transducer 14 moves within the pocket 12.

In embodiments where at least a portion of one of the pockets 12 is rigid, the rigid portion of the pocket may be formed from a variety of materials such as metals, plastics, ceramics and combinations of such materials. In some embodiments, at least a portion of the rigid materials may be electrically conductive or have applied to selected areas of such materials an electrically conductive coating. Conductive inks may be applied to a substrate to enable power to be transmitted from the power supply to the piezoelectric material.

In additional embodiments where at least a portion of the pocket is very flexible, the flexible portions may be formed of woven materials, nonwoven materials, films, or combinations thereof. The term “nonwoven web” generally refers to a web having a structure of individual fibers or threads which are interlaid, but not in an identifiable manner as in a knitted fabric, which would be generally referred to as a “woven web”. Examples of suitable nonwoven fabrics or webs include, but are not limited to, meltblown webs, spunbond webs, carded webs, hydroentangled webs, etc. The basis weight of the nonwoven web may generally vary, such as from about 0.1 grams per square meter (“gsm”) to 120 gsm, in some embodiments from about 0.5 gsm to about 70 gsm, and in some embodiments, from about 1 gsm to about 35 gsm.

The flexible layers may include elastomeric or extensible portions to enhance their performance. The term “elastomeric” and “elastic” refers to a material that, upon application of a stretching force, is stretchable in a direction (such as the machine direction or cross-machine direction), and which upon release of the stretching force, contracts/returns to approximately its original dimension. The term “extensible” generally refers to a material that stretches or extends in the direction of an applied force by at least about 50% of its relaxed length or width. An extensible material does not necessarily have recovery properties.

These materials may be electrically conductive or may include portions which are electrically conductive, such as, for example, conductive nonwoven webs. The term “conductive nonwoven web” generally refers to a nonwoven web which is capable of conducting an electric current. Suitable conductive nonwoven webs include conductive fibers of carbon, such as SGL C25, available from Technical Fibre Articles Ltd. Acrylonitrile or pitch fibers may also be used. The conductive nonwoven web may include a variety of fibers, not all of which are conductive.

Conductive fibers useful in fibrous webs include carbon fibers and metallic fibers. Suitable carbon fibers include fibers made entirely from carbon or fibers which contain only enough carbon so that the fibers are electrically conductive. Carbon fibers may be used that are formed from a polyacrylonitrile (PAN) polymer. Such carbon fibers are formed by heating, oxidizing, and carbonizing PAN polymer fibers. PAN-based carbon fibers are widely available from companies such as Toho Tenax America, Inc. of Rockwood, Tenn. Other raw materials used to make carbon fibers include rayon and petroleum pitch. Suitable conductive fibrous webs which include conductive fibers of carbon, such as SGL C25, are available from Technical Fibre Products Ltd. (Newburgh, N.Y.).

Suitable metallic fibers may include silver, copper and aluminum fibers and so forth. Such conductive fibers can have a variety of suitable lengths and diameters. Conductive polymeric fibers may be used and include fibers made from conductive polymers as well as polymeric fibers containing a conductive material or impregnated with a conductive material. Metal coated polymeric fibers and mixtures of these various conductive fibers may also be useful in the present invention. For example, U.S. Pat. Nos. 5,316,837 and 5,656,355, both to Cohen, disclose stretchable metalized nonwoven webs.

The conductive fibers may be combined with other fibers such as natural or synthetic cellulosic fibers including, but not limited to cotton, abaca, flax, esparto grass, straw, jute hemp, or fibers obtained from deciduous and coniferous trees, including softwood fibers or hardwood fibers. Synthetic fibers such as rayon, polyolefin fibers, polyester fibers, polyvinyl alcohol fibers, bicomponent sheath-core fibers, multi-component binder fibers, and the like may also be combined with the conductive fibers. Recycled fibers may also be used in combination with the conductive and non-conductive fibers. The amount of conductive fibers within the web may be selected based on various design criteria, such as the type of fiber and the end use of the web.

The conductive web may contain a substantial amount of pulp fibers and can be made using a tissue making process. For instance, in one embodiment, the conductive fibers can be combined with pulp fibers and water to form an aqueous suspension of fibers that is then deposited onto a porous surface for forming a conductive tissue web. The conductivity of such a web can be controlled by selecting particular conductive fibers, locating the fibers at particular locations within the web and by controlling various other factors and variables. For example, the conductive fibers can be incorporated into a web that includes non-conductive fibers such that the web is electrically conductive in a variety of zones, which may also form electrically conductive pathways. As such, the fibrous web can be made so that it is capable of carrying an electric current in the machine direction or cross-machine direction, or in any suitable combination of directions. The conductivity of the fibrous web can vary depending upon the type of conductive fibers incorporated into the web, the amount of conductive fibers incorporated into the web, and the manner in which the conductive fibers are positioned, concentrated or oriented in the web.

The amount of conductive fibers contained in the nonwoven web can vary based on many different factors, such as the type of conductive fiber incorporated into the web and the ultimate end use of the web. The conductive fibers may be incorporated into the nonwoven web, for instance, in an amount from about 1% by weight to about 90% by weight, or even greater. For instance, the conductive fibers can be present in the nonwoven web in an amount from about 3% by weight to about 60% by weight, such as from about 3% by weight to about 20% by weight.

A variety of binders including water and organic soluble polymers may be utilized to bind the various fibers into a web. If desired, the binders may be electrically conductive. Such binders are widely available and commonly known.

In some embodiments, electrospinning may be used to selectively apply conductive nanofibers to a nonconductive material such as a nonwoven material, a woven material or a film. Electrospinning refers to a technology which produces fibers from a polymer solution or polymer melt using interactions between fluid dynamics, electrically charged surfaces and electrically charged liquids. In general, a typical electrospinning apparatus useful for spinning nanofibers from a polymer solution includes a spinneret such as a metallic needle, a syringe and syringe pump, a high-voltage power supply, and a metal collector which is grounded. The polymer solution, which typically includes polymer and a solvent, has been loaded into the syringe and is driven to the needle tip by the syringe pump so that a droplet is formed at the needle tip. An electrode such as a stainless steel wire may be positioned within the syringe and may be used to charge the polymer solution. When the polymer solution within the syringe is charged, the droplet is drawn toward the grounded collector and stretched into a configuration commonly known as a Taylor cone. As the jet of solution flows from the needle tip to the grounded collector, the jet is stretched and the solvent in the polymer solution evaporates. As the jet of solution approaches the grounded collector, the electrical forces cause a whipping affect which results in the nanofibers being spread out onto the collector. A material, such as a nonwoven web, may be positioned between the collector and the tip of the needle to collect the nanofibers.

Many publications are available which describe fully the electrospinning process and its controlling variables, such as, for example, solution viscosity, the distance between the spinneret tip and the collector, voltage and solution conductivity.

For pockets which are formed independently of a support member, the pockets may be adhered in any manner or pattern to a support layer. In some embodiments, the pockets 12 may be adhered directly to the skin of the user by a wave transmission layer such as an adhesive or hydrogel. In such embodiments, the power supply may be attached to or otherwise integrated with the pocket 12, resulting in a convenient and portable ultrasound therapy article which can be easily adjusted by a user.

The ultrasound transducer 20 is movable within the pocket 12, and may occupy only a small portion of the interior of the pocket. For example and as shown in FIG. 4, the ultrasound transducer 20 may contact the upper and lower interior surfaces of the pocket 12 if such contact provides the electrical connection necessary to power the ultrasound transducer 20. In other embodiments, the ultrasound transducer 20 may not extend through the full height of the pocket 12. The ultrasound transducer 20 similarly may take up significantly less than the full width of the pocket 12. The ratio of the width of the ultrasound transducer 20 to the width of the pocket 12 may vary from 1% to 85%, depending on the amount of transducer movement that is desired. To achieve the required movement of the ultrasound transducer 20 in the pocket 12, the ultrasound transducer 20 and pocket 12 may be variously shaped. For example, the ultrasound transducer 20 may be circular, octagonal, oval or square in cross-section, although other cross-sectional shapes may be used. The pocket 12 may have a shape similar to or dissimilar to the ultrasound transducer 20.

It may be preferred that the layers of the article positioned between the transducer 14 and the user do not significantly interfere with or undesirably dissipate the generated ultrasound waves. In selected embodiments, the enclosing layer may include features which substantially inhibit the passage of the generated ultrasound waves. In such embodiments, the support layer should have a resistance to the ultrasound waves which is less than the resistance of the enclosing layer or walls. The resistance could be increased by adding additional basis weight to a layer or adding additional reinforcing layers such as, for example, an additional layer of nonwoven or scrim. This may provide increased resistance to ultrasound waves while maintaining flexibility. It is preferred that the article is configured so that the ultrasound waves encounter a lower resistance to entering the user's tissue than the higher resistance presented by the enclosing layer or other structures of the present invention.

As shown in FIGS. 3 and 4, the ultrasound transducer may include a first electrode 16, a second electrode 18 and a piezoelectric material 14 disposed between the electrodes 16 and 18. In selected embodiments, the piezoelectric material 14 may be in direct contact with the electrically conductive portions of the pocket 12. A power supply (not shown) is provided and is in electrical communication with the first and second electrodes 16, 18 so that, when power is supplied to the electrodes the piezoelectric material generates appropriate levels of ultrasound waves. These ultrasound waves then pass through the article and into the user for therapeutic benefit. A variety of suitable ultrasound transducers are available for use in the present invention. For example, piezoelectric fibers made from lead zirconate titanate and epoxy, and piezoelectric fiber multilayer composites may be used and obtained from Advanced Cerametrics Inc. (Lambertville, N.J.), Smart Materials Co. (Sarasota, Fla.) and Piezo Technologies (Indianapolis, Ind.).

There are a variety of ways in which power can be supplied to the ultrasound transducer 12. While the manner of connection may vary, it should not significantly limit the movement of the ultrasound transducer 14 within the pocket 12. The power supply may be portable to enable a user to move about freely while utilizing the article of the present invention. The power supply is preferably small enough so that it may be attached in a convenient and simple manner to the support layer 32 or the pocket 12 and permit the user to move about as they desire. The power supply may include a switch to enable a user to turn the power on and off as desired.

In some embodiments, the power supply may include a battery, a frequency source and a control circuit for frequency and amplitude. The power supply may also include an amplifier to elevate the mechanical energy generated from the piezoelectric material to a frequency range of about 20 kH to about 5 MHz and an intensity range of about 0.03-2 W/cm². Different battery configurations may be used with the present invention. For example, a single 12V battery may be utilized and can be obtained from a variety of sources, including Nexergy (Columbus, Ohio). If desired, two batteries may be used to drive an AC circuit. Cascaded batteries and push-pull transistor arrangements can provide a time-varying source of power to the ultrasound transducer. DDS waveform generators may be utilized with the present invention, and are available from Analog Devices, Inc. (Norwood, Mass.). Similarly, a variety of commercially available inverters are suitable for use with the present invention and include inverters available from DC/AC Power Inverters (Wilmington, N.C.) that can supply a pure sine wave product that converts 12V DC to 120V AC. Additionally, a linear amplifier that is able to appropriately elevate the frequency from 60 Hz to 1-5 MHz is available from Hotek Technologies (Tacoma, Wash.)

In some embodiments, the power supply may be directly connected via flexible wires to each of the electrodes 16, 18, the wires extending through the pocket 12. Alternately, the electrodes 16, 18 or the piezoelectric material 14 may be in sliding electrical contact with electrically conductive interior surfaces of the pocket 12. In such an embodiment, the electrically conductive regions 26, 28 are connected to the power supply. The connection may be through electrically conductive pathways applied to the substrate such as a film, woven or nonwoven web or a somewhat flexible molded plastic shape. In some embodiments, the conductive pathways may be directly formed into the material, such as by utilizing conductive fibers in strategically positioned locations in a woven web.

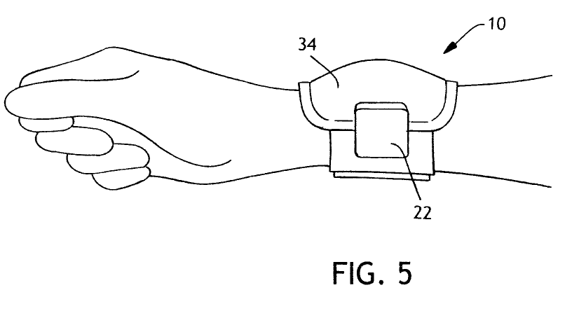

The article 10 may also include portions which extend outwardly and function to wrap around or otherwise attach to a portion of a user, such as an arm, leg, hand, foot, and the like. As seen in FIG. 5, the article may be attached to a user by wrapping the article around the user's wrist. The article 10 is shown with a power supply 22 attached directly to the enclosing layer 34. The article may be secured to a user or about a portion of a user's body by any available attachment scheme, including adhesive, hook and loop fasteners, clips, pins or ties and the like.

A hydrogel layer on the user facing side of the article may function as a coupling layer or wave transmission layer to assist the conduction of the ultrasound waves from the material into the body.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An article comprising:
at least one pocket;
an ultrasound transducer positioned within the pocket and including a piezoelectric material; and
a power supply in electrical communication with the piezoelectric material;
wherein the ultrasound transducer is movable within the pocket.

2. The article of claim 1, the ultrasound transducer further including a first and second electrode, the piezoelectric material being disposed therebetween, the power supply in electrical communication with the first and second electrodes.

3. The article of claim 1, the pocket including interior surfaces, a portion of the interior surfaces being electrically conductive, the ultrasound transducer and the power supply in electrical communication with the electrically conductive interior surfaces of the pocket.

4. The article of claim 3, the ultrasound transducer further including a first and second electrode, the piezoelectric material being disposed therebetween, the first and second electrodes in electrical communication with the electrically conductive interior surfaces of the pocket.

5. The article of claim 1 further including a wave transmission layer positioned between the ultrasound transducer and a user.

6. The article of claim 5 wherein the wave transmission layer is formed of a hydrogel material.

7. The article of claim 5 wherein the wave transmission layer is adapted to adhere the article to the user's skin.

8. The article of claim 1 wherein the power supply includes a battery, a DC/AC inverter and an amplifier.

9. The article of claim 1 wherein at least a portion of the pocket is flexible.

10. The article of claim 1, further including a support layer and an enclosing layer joined to each other to form the pocket.

11. A therapeutic article comprising:
a support layer including at least one electrically conductive region;
an enclosing layer including at least one electrically conductive region, the enclosing layer being joined to the support layer to form at least one pocket including the electrically conductive regions within the interior of the pocket,
an ultrasound transducer positioned within the pocket and in electrical communication with the electrically conductive regions of the enclosing layer and the support layer;
a power supply in electrical communication with the electrically conductive regions of the support layer and the enclosing layer;
wherein the ultrasound transducer is movable within the pocket.

12. The article of claim 11, the ultrasound transducer further including a first and second electrode, the piezoelectric material being disposed therebetween, the first and second electrodes in electrical communication with the electrically conductive regions of the enclosing layer and the support layer.

13. The article of claim 11 wherein the pocket is formed at least in part by a flexible material.

14. The article of claim 13 wherein the enclosing layer includes a nonwoven material.

15. The article of claim 11 further including an adhesive layer in contact with the support layer, the adhesive layer adapted to secure the article to a user.

16. A therapeutic article adapted to provide ultrasound energy to a user, the article comprising:
a wave transmission layer adapted to be positioned on the skin of a user;
a support layer including an upper surface and a lower surface, the upper surface including at least one electrically conductive region and at least a portion of the lower surface adhered to the wave transmission layer;
an enclosing layer including an upper surface and a lower surface, at least a portion of the lower surface of the enclosing layer being joined to at least a portion of the upper surface of the support layer to form at least one pocket, at least a portion of the lower surface of the enclosing layer including at least one electrically conductive region;
an ultrasound transducer positioned within at least one of the pockets and comprising a first and a second electrode and a piezoelectric material in contact with and positioned between the first and second electrode, the first electrode in electrical communication with at least a portion of the electrically conductive region of the support layer and the second electrode in electrical communication with at least a portion of the electrically conductive region of the enclosing layer,
a power source in electrical communication with the electrically conductive regions of the support layer and the enclosing layer;
wherein the ultrasound transducer is slidable within the pocket.

17. The article of claim 16, wherein the wave transmission layer includes a hydrogel.

18. The article of claim 16, the support layer being formed of a flexible material.

19. The article of claim 16, the enclosing layer being formed of a flexible material.

20. The article of claim 16, the enclosing layer being formed from a flexible electrically conductive material.
